(19) United States
(12) Patent Application Publication
(10) Pub. No.: US 2011/0166106 A1
(43) Pub. Date: Jul. 7, 2011

(54) ARTEMISININ AND DERIVATIVES THEREOF AS ANTIVIRALS

(76) Inventors: Manfred Marschall, Santa Barbara, CA (US); Jens Milbradt, Santa Barbara, CA (US); Dharam Ablashi, Santa Barbara, CA (US)

(21) Appl. No.: 12/999,885

(22) PCT Filed: Jun. 17, 2009
(86) PCT No.: PCT/US09/47712

§ 371 Date: Mar. 17, 2011

Related U.S. Application Data
(60) Provisional application No. 61/073,341, filed on Jun. 17, 2008.

(51) Int. Cl. A61K 31/675; A61K 31/352; A61K 31/522; A61K 31/4184; A61K 31/662; A61P 31/12

(52) U.S. Cl. 514/86; 514/450; 514/263.38; 514/395; 514/120

(57) ABSTRACT

Methods to treat various herpes viral infections using the natural product artemisinin and derivatives of that compound are described. The methods are especially applicable for treatment of conditions associated with HHV-6, and are also applicable to the treatment of conditions that are induced or exacerbated by an HHV-6 infection or by a reactivation of a latent stage of an HHV-6 infection.

### TECHNICAL FIELD

This invention relates to a natural product commonly referred to as artemisinin and certain of its derivatives, which have been shown to provide antiviral activity against several different viruses. The invention provides compositions and methods useful for the treatment of herpes viral infections, including HHV-6 infections and drug-resistant viral infections, as well as treatment of disorders that are associated with or induced or exacerbated by viral proteins or viral activity.

### BACKGROUND ART

Artemisinin (1) is a natural product that is used to treat malaria.

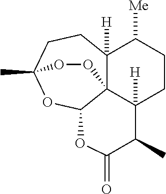

1: Artemisinin

Artemisinin is derived from a plant that was recognized in traditional Chinese medicine as useful for treating various conditions, particularly malaria. The isolated active ingredient from that plant (called sweet wormwood or qinghaosu (Artemisia annua L.)), artemisinin, is now a preferred treatment for malaria throughout much of the world. It affects the major malarial parasites (Plasmodium falciparum and Plasmodium vivax), and is effective at a very early stage of infection. While the dosage required to treat malaria is relatively high, it has not produced many serious adverse effects or induced significant drug resistance.

Extensive research has been done to understand its effects on malaria, and to develop derivatives or analogs of artemisinin that have better activity or pharmacokinetic properties for convenient oral administration. Among the most widely used of these is artesunate (2e), which is believed to be a prodrug for the active form of artemisinin. The artemisinin derivatives are commonly administered in combination with other effective antimalarial drugs: this is done to prevent development of resistance. So far, these compounds are among the best treatments for malaria that is resistant to other drugs, and these combination treatments are recommended in order to avoid or at least delay the appearance of malaria strains that are resistant to the artemisinins. E. A. Ashley, et al., Clin. Infectious Diseases, vol. 41, 425-32 (August 2005). Because they have not produced resistant malarial strains and are potentially very cost-effective, the artemisinin derivatives are badly needed in the fight against malaria in less developed countries where malaria is most prevalent.

Selected Derivatives or Analogs of Artemisinin

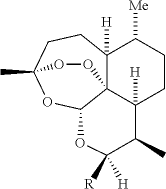

2: Analogs of Artemisinin
a: R═OH (dihydroartemisinin)
b: R═OMe (artemether)
c: R═OEt (artemotil)
d: R═OCH₂-(p-Carboxyphenyl) (artelinic acid)
e: R═OC(O)CH₂CH₂COOH (artesunic acid or artesunate)

The active species from all of these derivatives is believed to be dihydroartemisinin (2a), and the other structures (2c-2e) may provide more suitable delivery or stability properties, probably by protecting the somewhat acid-labile acetal structure of 2a.

Some derivatives of artemisinin have also been found to possess anticancer effects. For example, T. Efferth, et al., Free Radical Biology & Medicine, 37(7), 998-1009 (2004) discusses mechanisms that attempt to explain why artemisinin and artesunate appear to be more toxic to some tumor cells than to healthy cells, and conclude that the effect correlates with increased iron uptake or metabolism. Their results support a link between the oxidative ‘peroxide’ linkage in these compounds and their biological effects, which they explain based on levels of mitochondrial aconitase and ferroxidase in certain types of tumor cells.

Some reports of antiviral activity for certain artemisinin derivatives have also appeared. Artesunate possesses anti-viral activity, primarily demonstrated as activity against human cytomegalovirus (HCMV) and herpes simplex (HSV-1), which is detectable in vitro (cell culture systems; Efferth et al., J. Mol. Med. vol. 80, 233-42 (2002)), and affects drug-resistant as well as wild type strains of HCMV, with lesser effects on influenza and HIV viruses. This activity of artesunate was also demonstrated in vivo (Kaptein et al, Antiviral Research vol. 69, 60-69 (2006)), where it was observable in rat models and was enhanced by co-administration with known antivirals such as ganciclovir or with ferrous iron, though the activity of artemisinin in that report was described as ‘relatively low’. Finally, artesunate was very recently reported to be a potent antiviral in a patient having advanced stage drug-resistant cytomegalovirus infection, following hematopoietic stem cell transplantation (Shapira et al., Clinical Infectious Diseases vol. 46, 1455-57 (2008)).

It has now been discovered that artemisinin and its derivatives are useful for treating various viral infections, particularly certain human herpes virus infections (e.g., HHV-1 through HHV-8, particularly HHV-6) and conditions associated with viral proteins or viral activity.

Human herpes viruses (HHV-1 through HHV-8) are major pathogens of man. Primary acute infections with these viruses as well as lifelong persistent infections of the host can eventually cause severe pathological consequences which, under unfavourable immunological circumstances, can lead to life threatening clinical manifestations. At present, clinically available drugs for anti-herpes viral therapy are primarily composed of nucleotide/nucleoside or non-nucleotide inhibitors of viral DNA synthesis. The clinical applications of these drugs, however, face severe limitations due to adverse effects on the patient and resistance development by the virus, so novel antiviral strategies are needed.

In the case of roseoloviruses (human herpesvirus 6 (HHV-6) and HHV-7), a novel approach for antiviral therapy has been demonstrated, using artesunate or other artemisinin derivatives. HHV-6 was discovered in 1986 (Salahuddin, et al., Science, vol. 234, 596-601 (1986)), and was reported to be related to HCMV based on sequence homology. G. L. Lawrence, J. Virology vol. 64(1), 287-99 (1990). HHV-6 is very common in humans: most humans (over 97%) have antibodies to at least one form of HHV-6 from an early age. Id. However, it has not been thoroughly studied partly because most HHV-6 infections produce little symptomology, and its importance in a variety of disease conditions has only recently begun to be understood. HHV-7 is another recently identified virus that, like HHV-6, frequently infects infants and causes the childhood disease roseola.

There are two recognized variants of HHV-6, HHV-6A and HHV-6B. HHV-6A is the strain most likely to be found in MS, CFS, AIDS and cancer patients. HHV-6B infects close to 100% of children by the age of two, causing mild flu-like symptoms and rash in some and little symptomology in others; it occasionally progresses to cause high fever, encephalitis and seizures. Ablashi, et al., Arch. Virol., vol. 29, 363-66 (1993); Yamanishi, et al., Lancet, 1065-69 (1988). It has been shown to invade the CNS, where it can enter latency and reactivate periodically. HHV-6B causes roseola, febrile illnesses and encephalitis in infants and reactivates in transplant patients, causing complications such as encephalitis, pneumonitis and liver failure. In most cases, the virus goes into latency after a primary infection stage. However, particularly in patients with impaired immune function, the virus may persist in its active state at low levels for years and then become reactivated, with potentially life-threatening consequences. Recent studies strongly associate this reactivation with a variety of other disorders, mainly of the central nervous system (CNS).

Human Herpes virus 6 (HHV-6) is an immunosuppressive and neurotropic virus that can cause encephalitis and seizures during a primary infection or when reactivated from latency in immunosuppressed patients. Primary infection with HHV-6B leads to exanthem subitum (sudden rash) and febrile illness. New research suggests that HHV-6 may play a role in several chronic neurological conditions including MS (multiple sclerosis), mesial temporal lobe epilepsy (MTLE), status epilepticus and chronic fatigue syndrome (CFS). See, e.g., Yoshikawa et al., J. Med. Virology, vol. 66, 497-505 (2002). Its association with MS is demonstrated by the frequent presence of HHV-6 antigens and DNA in MS patients at levels suggesting active HHV-6 infections. Id. It is postulated that HHV-6 induces or accelerates CNS disorder progression by eliciting cytokine production, particularly IL-1beta, IL-6, and TNF-alpha.

While it is generally known that HHV-6 causes roseola infantum, febrile illness, infectious mononucleosis, and occasional seizures and encephalitis in infants, most physicians do not realize that HHV-6 can persist in a subacute form causing CNS dysfunction. HHV-6 can also cause selective immune suppression and alterations in cytokines that make it more difficult for the body to fend off cancer, intracellular pathogens, viruses and mycobacteria. Finally, HHV-6 has potent transactivating properties that cause it to stimulate other viruses, such as EBV, CMV, HHV-8, HIV, and HERV-K18.

HHV-6 is remarkably widespread as a latent infection, and it can even be integrated into a person's own DNA. Such congenital infection has been detected in a small percentage of the population, who carry at least a portion of the HHV-6 genome in all of their cells. Congenital infection and post-natal infection with HHV-6B can be followed by later infection with HHV-6A. Hall, et al., Clinic. Inf. Disease, 26:132-37 (1998); DiLuca, et al., J. Inf. Disease, 70:211-25 (1994). The HHV-6 genome contains over 100 open reading frames (ORFs), so it encodes many proteins. Mori, et al., PERSPECTIVES IN MEDICAL VIROLOGY, pp. 47-57, Krueger and Ablashi, eds., Elsevier Press (2006). Certain of its proteins are characteristic of various stages of infection. For example, within 8 hours after infection, one can detect a protein called “immediate early protein”, or IE1 protein; shortly thereafter, one can detect additional proteins called IE2 proteins. IE2may promote the expression of other essential HHV-6 proteins and accelerate infection. Id. Various IE proteins have been implicated in maintaining latency of HHV-6, and also appear to be related to the cytokine inducing effect of HHV-6. Yoshikawa, et al., at 504.

Whether directly or via its effect on cytokines, HHV-6 appears to be closely associated with various CNS conditions. Id. See also Fotheringham, et al., J. Neuroimmune Pharmacol., vol. 3, 105-116 (2008). Reactivation of latent HHV-6 is associated with certain neurological disorders, including epilepsy and multiple sclerosis (MS), possibly due to dysregulation of glutamate uptake in HHV-6 infected cells. Fotheringham, et al. This dysregulation can cause either increased glutamate uptake (found with acutely infected astrocytes) or decreased glutamate uptake, which occurs in chronically infected cells. Id. Dysregulation is posited as a mechanism whereby a latent HHV-6 infection can, upon reactivation, induce various CNS disorders including MS and epilepsy. The treatments described herein can inhibit the reactivation of a latent HHV-6 infection, and thus can be used to not only protect against reactivation of HHV-6 (e.g., encephalopathy caused by HHV-6 acute infection), but also to protect a subject at risk for neurological disorders that can be triggered by such HHV-6 reactivation. Such disorders include epilepsy, MS, chronic fatigue syndrome (CFS), depressive disorder, myocarditis, drug-induced hypersensitivity syndrome (DIHS), psychiatric disorders, and bipolar disorder, in addition to HHV-6 encephalopathy caused by the HHV-6 primary infection. Kobayashi, et al., Abstract 15-2, 6^{th} International Conf. on HHV-6 and HHV-7, Baltimore, Md. (June 2008). Strong evidence of a link between reactivation of HHV-6 infection and multiple sclerosis has been shown. Chapenko, for example, concludes that HHV-6 reactivation “is implicated in exacerbation of MS.” J. Med. Virology, 69:111-17 (2003). One aspect of the invention is the treatment of latent HHV-6 infection to prevent reactivation and the adverse neurological disorders that can result from such reactivation. Another aspect of the invention includes treatment of these secondary conditions associated with reactivation of HHV-6 infection.

There is also a strong association between Epstein-Barr Virus (EBV) and multiple sclerosis, and the compounds of formula (3) are also active against EBV. Lunemann, et al., Trends Immunol., 30(6): 243-48 (May 2009). Consequently, in one aspect the invention provides a method to use such compounds to treat EBV or to treat or prevent disorders such as MS that may be caused by an EBV infection. A subject having markers associated with EBV, such as antibodies or proteins detectable in plasma, can be identified by routine methods, and treatment of such subjects with artemisinin-like compounds as described herein can be used to reduce the risk of developing MS in such subjects.

Similarly, HSV-1 infections, HSV-2 infections, and VZV infections can enter a latent stage and become reactivated, such as in an immunocompromised host, e.g., a transplant patient receiving immunosuppressant drugs. See, e.g., Berger et al., Arch. Neurol. 65(5): 596-600 (2008). Indeed, like HHV-6, these viruses can be found in surprisingly large fractions of the population in a latent stage, and their reactivation under certain circumstances can have grave consequences. In such subjects, the infecting virus can reactivate, and, like HHV-6, the reactivation can cause complications such as certain cancers, increased susceptibility to neurologic disorders, etc., as well as more devastating primary infection symptoms that increase complications and risk of organ rejection if the subject is a transplant recipient. These viruses are also treatable with the artemisinins disclosed herein. Thus in another aspect, the invention provides a method to treat HSV-1, HSV-2, and VZV infections, or to inhibit or prevent a reactivation of a latent infection with one of these viruses. Suitable subjects for such treatment can be identified by the same methods described herein for identifying subjects at risk for reactivation of HHV-6, e.g., detection of elevated levels of antibodies to the particular virus, or expression of proteins or nucleic acids associated with the underlying virus. The treatment methods and preferred compounds for these aspects of the invention are the same as those described herein in greater detail in connection with HHV-6.

Unfortunately, chronic HHV-6 infections are notoriously difficult to detect with current diagnostic tests. Molecular assays (DNA PCR tests) are useful for detection of acute infections but are not sensitive enough to detect chronic infection because the virus remains active only in selected tissues (often the central nervous system). This means there is often very little free virus circulating in the serum or plasma to be detected by typical blood tests. Serological assays (tests that look for antibodies instead of virus) are more sensitive but it is difficult to differentiate between active and latent infections using these. While these antibodies fall gradually after an infection, they can persist at high levels for several years before falling to lower levels. This is further complicated by the fact that most humans are exposed to HHV-6B at an early age, and thus can express antibodies to it as a result of such infection or residual latent infection, since HHV-6 typically remains present in latent form after a primary infection resolves. Also, serological assays cannot necessarily differentiate between the A and B variants. In spite of the fact that these antibody tests are imperfect, elevated antibodies may be a patient's only clue that a chronic infection is ongoing in the CNS, cardiac or other tissues.

Because HHV-6 is so widespread, any subject may have at least a low level of antibodies reactive toward HHV-6 antigens. This finding complicates research related to understanding the effects of HHV-6 infection as well as diagnosis of conditions that are associated with an HHV-6 viral infection, protein or activity. While an HHV-6 antibody titer of 1:1280, 1:640 or 1:320 in a child or adolescent might be perfectly normal, a titer of this level is rare in an otherwise healthy adult(depending upon the lab or test methodology), and could be a sign of chronic infection in an adult with clinical symptoms. In some embodiments, HHV-6 antibody titer is used to select a subject for treatment with the methods disclosed herein, and/or to monitor the effectiveness of treatment over time—recognizing that antibody titer will not drop precipitously even if an underlying infection is controlled or eliminated.

Preferably, an antibody titer for such a diagnosis or evaluation will be measured using IFA (indirect fluorescent antibody) methods, which are more quantitative and reliable than ELISA, and preferably the titer selected as a cut-off value will be at least one dilution (2×) above the median for healthy subjects similar to the patient. In some embodiments, the cut-off will be two dilutions (4×) above a median value for healthy subjects. For example, a subject may be identified as suitable for treatment with the methods disclosed herein when his or her antibody titer is in the top 25% range for the subject's cohort; or within the top 15% range for the subject's cohort. In certain instances, the subject is diagnosed as suitable for treatment based on antibody titer if the subject's antibody titer is at least one dilution (i.e., 2×) higher than that for age and gender-matched healthy subjects.

HHV-6 is now being studied very extensively, as researchers are beginning to appreciate that it is not only widespread, but is also closely associated with a diverse and surprising array of conditions. As discussed above, it is associated with MS and epilepsy. In addition, it is the most common cause of mental confusion in post-transplant patients, presumably due to reactivation during treatment with immune suppressants. HHV-6 limbic encephalitis occurs in approximately 4% of transplant patients resulting in intermittent confusion, poor coordination, flat affect and somnolence.

Febrile seizures (“FS”), encephalitis, and encephalopathy have been recognized as major neurological complications of primary HHV-6 infection. In addition, these neurological complications may also be associated with reactivation of the latent virus in the CNS. Hall, C B et al. Human herpesvirus-6 infection in children. A prospective study of complications and reactivation. N. Engl. J. Med. 1994; 331:432-438; Jones, C M. Acute encephalopathy and status epilepticus associated with human herpes virus 6 infection. Dev. Med. Child Neurol. 1994; 36:646-650. Consequently, these symptoms especially if found in connection with evidence of the presence of HHV-6 virus or antibodies, may indicate a need for treatment such as those described herein for the control of HHV-6 infection or activity, especially if theses symptoms are found in connection with evidence of the presence of HHV-6 virus or antibodies.

In a prospective study of the consequences of prolonged febrile seizures (FEBSTAT), Leon Epstein and associates reported the frequency of human herpesvirus-6 (HHV-6) infection a cause of febrile status epilepticus (FSE) to be 29.7%, or 33 of 111 infants evaluated. Epstein, Leon International Conference on HHV-6 & 7, 2008, Baltimore, abstract. Asano et al reported that 8% of infants in Japan with FSE experienced convulsions. Asano Y et al. Clinical features of infants with primary herpesvirus-6 infection, Pediatrics 1994; 93:104-108. Hall et al noticed that 9.7% of infants and children under the age of 3 years in the USA with acute febrile illness displayed primary HHV-6 infection and 13% had seizures with primary HHV-6 infections.

Researchers have long known that viruses can cause seizures in infants, and viruses have been suspected in the pathogenesis of epilepsy but direct evidence was lacking, in part because there are no tests available to determine if a patient has an abortive infection in the central nervous system tissue. A paper by a Japanese researcher in 1993 suggested that HHV-6B plays a role in recurrent seizures, but the possibility of viral etiology was largely ignored. Kondo K, Nagafuji H, Hata A, Tomomori C, Yamanishi K., Association of human herpesvirus 6 infection of the central nervous system with recurrence of febrile convulsions, J Infect Dis. 1993 May; 167(5):1197-200. NINDS researcher Steve Jacobson looked at the resectioned brain tissue from 16 patients who had portions of their brain tissue removed as treatment for refractory MTLE. He found 11 of 16 patients with MTLE (but 0 of 7 control patients) had high loads of HHV-6B DNA. In a follow-up paper, Jacobson and colleagues at the NINDS demonstrated that as many as two thirds of patients with Mesial Temporal Lobe Epilepsy (MTLE) may in fact suffer from a chronic HHV-6B infection. Fotheringham J, et al., Association of human herpesvirus-6B with mesial temporal lobe epilepsy, PLoS Med. 2007 May; 4(5):e180. Jacobson's research suggests that HHV-6 causes a dysfunction in the astrocytes leading to the injury of the sensitive neurons in the hippocampus that trigger MTLE.

Research presented by Yale neurologist Nihal De Lanerolle at the 2008 International Conference on HHV-6 & 7 showed in sclerotic (MTLE) hippocampi the presence of HHV-6B virus not only predominantly in astrocytes but also in neurons (both pyramidal and granule cells) and around blood vessels in endothelial cells and presumptive macrophages. Virus was also detected in astrocytes and neurons in temporal neocortex. Virus was also found in some PTLE and MaTLE cases but at lower density. These observations confirmed the presence of HHV-6 virus in the epileptic brain tissue and suggested that it may enter the brain through the cerebral vasculature. De Lanerolle, Nihal, International Conference on HHV-6 & 7, Baltimore, 2008.

In a review of 416 seizure patients less than 3 years of age, 24% had primary HHV-6 infections. Millichap J G, Millichap J J, Influenza virus and febrile convulsions, J. Infect. Dis. 2004 Feb. 1; 189(3):564. Another study on the HHV-6 & 7 infections in hospital admissions in Britain and Ireland during the first two years of life found that 17% of the encephalitis cases were associated with primary infections of HHV-6 and 7, and that these two beta-herpesviruses were equally significant in these cases. Ward K N, Andrews N J, Verity C M, Miller E, Ross E M. Human herpesviruses-6 and -7 each cause significant neurological morbidity in Britain and Ireland. Another study in Japan found that of those patients with three or more seizures, 80% had evidence of HHV-6 in the cerebral spinal fluid compared with only 14% in patients with an isolated seizure. Kondo K, Nagafuji H, Hata A, Tomomori C, Yamanishi K. Association of human herpesvirus 6 infection of the central nervous system with recurrence of febrile convulsions. J. Infect. Dis. 1993 May; 167(5):1197-200. Another study in Japan found that HHV-6 can result in convulsions at the time of the rash outbreak following a primary infection with HHV-6. They called this condition HEEC or “human herpesvirus-6 encephalopathy with cluster of convulsions in eruptive stage”. Nagasawa T, Kimura I, Abe Y, Oka A. HHV-6 encephalopathy with cluster of convulsions during eruptive stage, Pediatr. Neurol. 2007. January; 36(1):61-3. This study strongly supports the idea that HHV-6 plays a vital role in the etiology of these CNS tissues.

It has also been reported that antivirals may be a viable and significant treatment option for cancer. Current research shows that HHV-6 may play a role in various cancers, including Hodgkin's lymphoma, pediatric gliomas, oral carcinoma and cervical cancer. Solid pediatric brain tumors are the second most common malignancy of childhood and the most common cause of death from childhood cancer. HHV-6 proteins have been detected in 30% of pediatric gliomas and have not been detected in any non-glial brain tumors. This is consistent with its involvement in the CNS generally, and demonstrates that it can invade the brain. Broccolo 2008 (http://www.scivee.tv/node/6828). Chen M, Wang H, Woodworth C D, Lusso P, Berneman Z, Kingma D et al. Detection of human herpesvirus 6 and human papillomavirus 16 in cervical carcinoma. 1994. Am J Path; 145,1509-15.

There is also a strong association between Epstein-Ban Virus (EBV) and certain proliferative disorders such as EBV-associated lymphoproliferative disorder (EBV-LPD) (Meijer, et al., Curr. Opin. Hematol. 15(6):576-85 (2008)) and lymphomas (Cohen, et al., Leuk. Lymphoma, 49 Suppl 1:27-34 (2008)), and the compounds of formula (3) are also active against EBV. Consequently, in one aspect the invention provides a method to use such compounds to treat EBV-associated proliferative disorders, such as EBV-LPD and lymphomas (Burkitt's lumphoma, Hodgkins lymphoma, peripheral T-cell lymphomas, angioimmunoblastic T-cell lymphoma, extrnodal nasal type natural killer/T-cell lymphoma) that may be caused by an EBV infection. A subject having markers associated with EBV, such as antibodies or proteins detectable in plasma, can be identified by routine methods, and treatment of such subjects with artemisinin-like compounds as described herein can be used to reduce the risk of developing proliferative disorders in such subjects.

HHV-6 replication was demonstrated by intense staining with mab gp116/64/54 in glial tumor cells compared to surrounding tissues or tissues from patients without glial tumors. This strong staining suggests viral replication. The presence or absence of HHV-6 in pediatric brain tumors is not predictive of survival, but treatment with antivirals to HHV-6 or immunotherapies is usually helpful to these patients who are positive for HHV-6.

Lymphoma. Hodgkin's lymphoma (HL) has been associated with Epstein Barr Virus (EBV) and is characterized by the appearance of large cells called Reed-Sternberg (RS) cells when lymph nodes are viewed under the microscope. Thirty to 50% of RS cells in lymph nodes of HL patients have been found to express antigens to HHV-6; some of these same patients express EBV in the same cells. Antibodies to HHV-6 are also found in lymphocyte-plasmocytes and histiocytic cells of the same patients. Many immunofluorescent assays done on the sera of HL and NHL have found a high percentage of patients with elevated HHV-6 antibodies when compared to normal controls, especially in patients that relapsed. Ranger-Rogez S, Lacroix A, Denis F, Bordessoule D. HHV-6 and the lympho-hemopoietic system. In: HUMAN HERPESVIRUS-6. Krueger, Kruger, Ablashi (eds). Elsevier Science Publishers B. V. 2005.

In a review of early studies trying to link HL and HHV-6, contradictory results were found depending on which patients were studied and the techniques used; nonetheless there seems to be at least a subset of HL being due to the involvement of HHV-6 (Ranger-Rogez 2005). In more recent preliminary studies done using immunohistochemistry (IHC), in situ hybridization and polymerase chain reaction (PCR), Hartmann showed 23% of the HL tested and 33% of Non-Hodgkin's Lymphoma (NHL) samples studied are positive for HHV-6 whereas comparable non-lymphoma samples had no signal for HHV-6. Duncan C, et al. Identification of microbial DNA in human cancer. BMC Medical Genomics. May 2009, 2:22. Hartmann, Dan 2008 http://www.scivee.tv/node/6824

In a similar study by Lacroix (2008), HHV-6 was found to be associated with many types of lymphoma: in B-cell lymphoma, 22% samples were positive, in T-cell and natural killer (NK) cell lymphoma 23% were positive, and in HL, 35% (40% of the these HL cases were positive for EBV). Lacroix, Aurelie 2008 http://www.scivee.tv/node/6826 When Lacroix probed 86 HL samples by PCR for HHV-6 (V22 gene) and EBV (EBNA-1 gene), she found that the viruses tended to be associated with specific variants of the disease. Lacroix A, Jaccard A, Rouzioux C, Piguet C, Petit B, Bourdessoule D et al. HHV-6 and EBV DNA quantitation in lymph nodes of 86 patients with Hodgkin's lymphoma. J. Med. Virol. 2007; 79, 1349-56. HHV-6 was found in 79% of all these cases, but in a HL form called nodular sclerosis, 61/73 (84%) were positive. Most of these cases were the B variant of HHV-6 and were sensitive to chemotherapy. In contrast, only 62% of the 86 HL samples were positive for EBV and 80% of these proved to be the mixed cellularity form of HL and were less sensitive to chemotherapy. Lacroix, et al used two antibodies for IHC, antibody to DR7 on the oncoprotein from HHV-6B and gp116/64/54, which detects viral replication.

Oral Carcinomas. Oral carcinoma is the eighth most common tumor in the developed world and is especially prevalent in areas of Asia and the Pacific Islands where chewing betel quid or tobacco is common. Carcinogens have been suspected as being a primary cause of oral carcinoma, but viruses may play a role as well. Yadav M, Arivananthan M, Kuma S. HHV-6 antigen and viral DNA detected in cervical cells from archived tissue using histochemical staining and hybridization. Clinical and Diagnostic Virology, 1996; 7, 23-33; Yadav M, Chandrashekran A, Vasudevan D M, Ablashi D V. Frequent detection of human herpesvirus 6 in oral carcinoma. JNCI. 1994; 86, 1792-4.

Fresh tumor tissues and paraffin embedded samples of the buccal cavity of patients from India and Malaysia with squamous cell oral carcinoma were examined by PCR and IHC plus sera was tested by immunofluorescence assay. Most of these samples were positive for HHV-6 and had very high titers when compared to extremely low titers of normal controls. When similar samples from patients with nasopharyngeal carcinoma were studied, they were negative for HHV-6. The authors conclude that HHV-6 acts as a co-factor in combination with carcinogens to cause oral carcinoma.

Cervical Cancer. HHV-6 has been found in 6 out of 72 cases of squamous cervical carcinoma and cervical intraepithelial neoplasias in association with human papillomaviruses (HPV) while no virus was present in the 30 biopsies tested from normal cervical tissue or non-cancerous cervical tissue (Chen 1994). Another study (Yadav 1996) found HHV-6 in approximately a third of cervical cancer specimens studied. At the 6^{th} International conference on HHV-6 and HHV-7, Broccolo (2008) also found HHV-6 and HHV-7 DNA in 39% of cervical cancer tissues he studied. Quantitative PCR showed that HHV-6 increased with the severity of the disease, this was particularly noticeable in high grade squamous interepithelial lesions (H-SIL). In studies with nude mice, it has been shown that HHV-6 interacts with E6 and E7 transforming genes of HPV increasing the severity of disease. This was corroborated by immunohistochemical studies for both early and late antigens of HHV-6 on similar tissues. Control tissues were negative for HHV-6 and 7. Broccolo (2008) also found that HHV-6 and HHV-7 can infect epithelial cells in culture allowing co-infection of cells with both HPV and HHV-6 or 7. These studies make a strong case for HHV-6 acting as a cofactor in cervical cancer.

Other Cancers. In May 2009, researchers at Duke University identified HHV-6 DNA in colorectal cancer using a new genome-wide approach (Duncan, 2009). The findings suggest that there may be a higher prevalence of colorectal cancer among individuals infected with HHV-6.

HHV-6 in Myocarditis: Researchers at the Robert Bosch Medical Center in Stuttgart, Germany recently demonstrated that HHV-6 is far more common in myocarditis than was previously believed. Not only did they find 35% or 31 out of 87 biopsies positive for HHV-6, they determined that patients with HHV-6 involvement progressed more frequently toward chronic heart failure. Furthermore, unlike patients with parvovirus B-19 (the most common virus found), patients with HHV-6 had no chest pain and often did not seek treatment until the late stages of disease. Mahrholdt H, Presentation, patterns of myocardial damage, and clinical course of viral myocarditis, Circulation. 2006 Oct. 10; 114(15):1581-90. Epub 2006 Oct. 2., Kuhl et al found in 2005 found HHV-6 in 23% of 172 myocarditis patient biopsies, and also found that when the virus cleared (which happened spontaneously in 44% of the cases) there was patient improvement in left ventricular ejection fraction, while in patients where the virus did not clear, there was progressive impairment. Kühl, Viral persistence in the myocardium is associated with progressive cardiac dysfunction, Circulation. 2005 Sep. 27; 112(13):1965-70. Epub 2005 Sep. 19. Caruso et al demonstrated that HHV-6 infects the aorta and endothelial cells in the heart, contributing to inflammation by producing proinflammatory cytokines. Caruso A, HHV-6 infects human aortic and heart microvascular endothelial cells, increasing their ability to secrete proinflammatory chemokines, J Med Virol. 2002 August; 67(4):528-33. HHV-6 infects endothelial cells of the aorta as well as veins and capillaries of the heart. Infection with HHV-6 appears to cause vascular endothelial damage that is worse than infection with CMV, and may cause thrombotic microangiopathy. Takatsuka H, Bone Marrow Transplant. 2003 March; 31(6):475-9. There have been case reports of HHV-6 in large vessel arteritis and in fulminant myocarditis. Fukae S, Intern Med. 2000 August; 39(8):632-6. “A fatal case of fulminant myocarditis with human herpesvirus-6 infection.” Toyabe S, Clin Rheumatol. 2002 November; 21(6):528-32.

In 2008, Dirk Lassner and Uwe Kuhl of the Charity Medical University of Berlin reported that reactivation of HHV-6 genotypes A and B was frequently detected in the myocardium of patients with heart failure symptoms and that viral markers correlated with the symptoms and hemodynamic presentation of the patients. They evaluated the prevalence and course of a HHV-6 reactivation in 1656 baseline and 92 follow-up endomyocardial biopsies (EMB) of consecutive patients with clinically suspected acquired cardiomyopathies or persisting heart failure symptoms by nested-PCR. They identified 273 patients (16.5%) with cardiac HHV-6 infection/reactivation, including 144 tissue samples with multiple viral infections (62.3%). Electron microscopic analysis demonstrated HHV-6 viral particles in cardiac vascular endothelial cells, occasionally also in macrophages and in degenerating cardiac myocytes. While HHV-6B was located in vascular endothelial cells, HHV-6A was also seen in cardiomyocytes. Detection of mature herpes virions in cardiomyocytes by EM suggests a possible replication of virus in myocardial tissue. 2008 International Conference on HHV-6 & 7, Human herpesvirus-6 subtypes in patients with acquired cardiomyopathies and heart failure symptoms, by Dirk Lassner & U. Kuhl. Thus many cases of myocarditis may be treatable with the compounds, compositions and methods disclosed herein; particularly suitable subjects for such treatment may be selected based on detection of HHV-6 viral infection, or antibodies to HHV-6 proteins.

HHV-6 & Transplant Reactivation. HHV-6 reactivates in transplant patients who are immunosuppressed and is a major source of morbidity and mortality. Yoshikawa T, Curr. Opin. Infect. Dis. 2003 December; 16(6):601-6. Central nervous system complaints include memory loss, fatigue and difficulties with cognitive processing, seizures, and post-transplant acute limbic encephalitis (PALE). Noguchi T, MR imaging of human herpesvirus-6 encephalopathy after hematopoietic stem cell transplantation in adults, AJNR Am J Neuroradiol. 2006 November-December; 27(10):2191-5. Zerr D M., J. Clin. Virol. 2006 December; 37 Suppl 1:S52-6. Seeley W W, Neurology, 2007 Jul. 10; 69(2):156-65. The compositions and methods of the invention can be used to treat complications associated with transplants that arise because of the reactivation of HHV-6 from a latent stage.

In kidney transplants, HHV-6 has also been associated with nephropathy. Chapenko S, J. Clin. Virol. 2009 Jun. 2. Association of HHV-6 and HHV-7 reactivation with the development of chronic allograft nephropathy. HHV-6 reactivation is a cause of major complications after allogeneic hematopoietic stem cell transplantation (HSCT), and has been associated with acute graft-versus-host Disease (aGVHD), allograft rejections, central nervous system dysfunction and increased mortality. de Pagter P J, Biol Blood Marrow Transplant. 2008 July; 14(7):831-9. “Human herpes virus 6 plasma DNA positivity after hematopoietic stem cell transplantation in children: an important risk factor for clinical outcome.”

Consequences of HHV-6 reactivation in liver transplant patients include bone marrow suppression, central nervous system dysfunction, pneumonitis, hepatitis, increased severity of graft host disease, increased incidence of fungal infections and higher incidence of allograft rejection. Abdel Massih R C, Human herpesvirus 6 infections after liver transplantation, World J Gastroenterol. 2009 Jun. 7; 15(21):2561-9. Yoshikawa T., Human herpesvirus-6 and -7 infections in transplantation, Pediatr Transplant. 2003 February; 7(1):11-7.

Encephalitis. HHV-6 encephalitis occurs primarily as a complication of transplantation in immunocompromised patients but has also been reported in patients who are immunocompetent. Crawford J R, Human herpesvirus 6 rhombencephalitis in immunocompetent children, J Child Neurol. 2007 November; 22(11):1260-8. Using sensitive diagnostic assays, researchers at the NINDS/NIH and the California Encephalitis Project found HHV-6 DNA and elevated antibody levels in the spinal fluid of 40% of 35 encephalitis cases of unknown etiology. Ann Neurol. 2009 March; 65(3):257-67. Ann Neurol. 2009 March; 65(3):235-7.

Katherine Ward at the Royal Free and University College Medical School in London recently published data on HHV-6 & 7 infections in hospital admissions in Britain and Ireland during the first two years of life (Ward et al, 2005) and found that 17% of the encephalitis cases were associated with primary infections of HHV-6 and 7. High levels of HHV-6 DNA in peripheral blood are associated with the development of CNS dysfunction in stem cell transplant patients. Mortality rate for HHV-6 encephalitis is high and surviving patients often have neurological damage. Ogata M, Correlations of HHV-6 viral load and plasma IL-6 concentration with HHV-6 encephalitis in allogeneic stem cell transplant recipients, Bone Marrow Transplant, 2009 May 25.

HHV-6 & Psychiatric Disease. Transplant physicians are aware that HHV-6 reactivation is often accompanied by CNS dysfunction including alterations in mood and psychiatric condition. Zen D M, Clin Infect Dis. 2005 Apr. 1; 40(7):932-40. Epub 2005 Mar. 2. Japanese virologist Kazuhiro Kondo reported at the 2008 International. Conference on HHV-6 & 7 that 53% of depression and 76% of bipolar depression patients exhibit an antibody to an intermediate latent HHV-6 protein associated with latent (non-replicating) HHV-6. Kondo, K, Identification of novel HHV-6 latent protein associated with mood disorders in CFS, depressive disorder, bipolar disorder and HHV-6 encephalopathy, 2008 International Conference on HHV-6 & 7, Baltimore, USA, Kondo reported that transfecting this new protein, called SITH-1 (Small Intermediate Stage Transcript of HHV-6), into nervous system cells called glial cells, resulted in greatly increased intracellular calcium levels. Increased intracellular calcium levels are believed to play an important role in psychological disorders and can contribute to cell death. Expressing the SITH protein though the use of an adenoviral vector in mice resulted in manic-like behavior. Further, he reported that a serological study indicated that 71% of CFS patients with psychological symptoms and none of the healthy controls possessed the antibody against the SITH-1 protein (p<0.0001). This strongly suggests that these conditions are induced or exacerbated by HHV-6 viral infection or progression of the infection from a latent to an active stage. In view of this, it is expected that treatment with ART and other artemisinin would delay these adverse effects associated with elevated SITH levels. Niebuhr D W, Schizophr Bull. 2008 November; 34(6):1182-8. Epub 2007 Dec. 21.

Investigators at the Johns Hopkins Stanley Research Institute and the Walter Reed Army Institute of Research found that military personnel have elevated antibodies to HHV-6 6-12 months before the initial onset of schizophrenia, with a hazard ratio of 1.41. Niebuhr D W, Results from a hypothesis generating case-control study: herpes family viruses and schizophrenia among military personnel, Schizophr Bull. 2008 November; 34(6):1182-8. Epub 2007 Dec. 21.

HHV-6, especially the A variant, also can cause immune suppression. Recent studies indicate that HHV-6 alters immune function by selectively blocking dendritic cell maturation and IL-12 p70 production. HHV-6 can cause an elevation in cytokines such as IL-6, contributing to encephalopathy in these patients. (Enoki 2006; Ogata, et al., Bone Marrow Transplantation, 1-8 (2009)). Because HHV-6 can occur in a latent stage where it is difficult to detect, it is believed that it is associated with and either causes or contributes to other conditions. Indeed, latent HHV-6 infections are associated with adverse effects, including production of proinflammatory cytokines. Yoshikawa, et al., J. Med. Virology vol. 66, 497-505 (2002). Reactivation of latent HHV-6 is also associated with various conditions, particularly tissue rejection in transplant patients. While HHV-6 is still poorly understood, the HHV-6 Foundation has identified studies that associate HHV-6 infections with at least some cases of encephalitis, CFS, MS, epilepsy (e.g., J. Fotheringham, et al., PLoS Medicine, vol. 4(5), e180 (2007). Brain tissue from 69% of mesial temporal lobe epilepsy patients contained HHV-6B, while none of seven control patients had detectable amounts), for example. Similar correlations exist with myocarditis, cancer, drug induced hypersensitivity, and amnesia, as well as complications from immune suppression and tissue rejection. In view of the many effects that a chronic or acute HHV-6 infection can have, new methods of treatment are needed.

### SUMMARY OF EMBODIMENTS

Artesunate is a well-tolerated drug approved for malaria therapy and, moreover, has an excellent safety profile demonstrated by extensive use as a malaria treatment. Artesunate activity against HHV-6 and other human herpes viruses was investigated, and the data demonstrate that artesunate can be used as a safe, novel antiviral drug for the treatment of these disorders, especially for treating HHV-6-associated diseases.

In one aspect, the invention provides methods to treat human herpes virus infections and disorders associated with or exacerbated by the presence of HHV6 or its proteins. The methods comprise administering an effective amount of a compound of formula (3) or a pharmaceutical composition containing such a compound of formula (3):

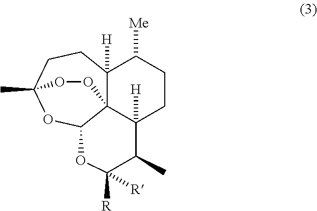

wherein R and R′ taken together form a carbonyl (═O),

or

wherein one of R and R′ is H,

and the other one of R and R′ is —OA,

wherein A is H or an optionally substituted group selected from alkyl, aryl, heteroaryl, arylalkyl, and heteroarylalkyl;

or a pharmaceutically acceptable salt or ester thereof.

In some embodiments of the invention, R′ in the compound of formula (3) is H. In some embodiments, the compound of formula (3) is selected from the group consisting of artemisinin, dihydroartemisinin, artemether, artemotil, artelinic acid, and artesunate.

In some embodiments, R′ is H and R is a substituted or unsubstituted alkyl.

In some embodiments where R or R′ comprises a carboxylic acid, the compounds of formula (3) are used as salts or esters of the carboxylic acid. In some embodiments, the ester is a simple alkyl ester such as a C1-C6 alkyl ester, where the C1-C6 alkyl is optionally substituted with one or more halo, hydroxyl, or C1-C4 alkoxy groups. Where the compound of formula (3) is an ester, it is sometimes a methyl or ethyl or propyl or butyl ester, or a 2-methoxyethyl ester or an ethylene glycol ester.

The antiviral compounds of formula (3) can be used to treat subjects afflicted with a variety of viruses. In some embodiments, the virus is a herpes virus. In some embodiments it is a virus such as HHV-1, HHV-2, HHV-3, HHV-4, HHV-5, HHV-6, HHV-7, or HHV-8. In some embodiments, the virus is a gamma-herpes virus (e.g., EBV, HHV-8) or an alpha-herpes virus (e.g., HHV-1, HHV-2, V2V); for example, the viral infection may be caused by a virus such as Epstein-Barr virus (EBV), HHV-8, HHV-3, Kaposi sarcoma associated herpesvirus (Ablashi, et al., Clin. Mirobiol. Rev., 439-64 (2002)), and varicella zoster virus (VZV). In some embodiments the virus is selected from beta-herpes group viruses such as roseoloviruses HHV-6 and HHV-7; for example, it may be HHV-6A or HHV-6B. Other specific conditions that can be treated by the methods of the invention include viral myocarditis, heart disease associated with a latent herpesvirus infection, hypersensitivity such as drug-induced hypersensitivity syndrome (DIHS) or DRESS (Drug Rash with Eosinophilia and Systemic Symptoms) that are associated with or exacerbated by a viral infection, autoimmune disorders, liver failure and hepatitis.

DIHS, or drug-induced hypersensitivity, is a rare but severe disease that includes multiorgan failure. Many different factors have been identified as causes, but the etiology remains unclear. However, HHV-6 has been shown to be associated with DIHS caused by carbamazepine hypersensitivity. Aihara, et al., British J. Dermatology, 149:165-69 (2003). Aihara demonstrates that reactivation of HHV-6 was associated with one example, along with a transient drop in gamma-globulin levels and elevated cytokine levels. Similarly, Kano, et al. demonstrated a strong association between hypersensitivity to anticonvulsant medications and HHV-6, showing HHV-6 reactivation as measured by surge in antibody titers in most patients exhibiting this hypersensitivity. Because DIHS is well correlated with HHV-6 reactivation, the compositions and methods of the invention are useful to treat, inhibit or prevent DIHS. Patients at risk for DIHS can be identified based on their HHV-6 antibody titers as discussed herein, and a compound of the invention can be administered prior to or along with a drug that is suspected of creating a risk for a DIHS reaction. Alternatively, a patient experiencing DIHS may be at increased risk of a reactivation of HHV-6 infection. Yoshikawa, et al., J. Clinical Virology, 37 Suppl 1: S92-S96 (2006). In this situation, a compound of formula (3) may be administered to the subject experiencing DIHS to prevent complications caused by onset of a reactivated HHV-6 infection, which worsens many symptoms of the initial DIHS reaction. Id.

This effect is similar in sequence and timing to the occurrence of HHV-6 reactivation in some transplant patients, who experience reactivation of latent HHV-6 infection a few weeks after starting immunosuppressive treatment. In each case, the subject can be recognized as at-risk for reactivation of HHV-6 based on the underlying event (drug treatment with an immunosuppressant or with an anticonvulsantor other drug associated with DIHS), combined with monitoring HHV-6 antibody titers to detect a surge in antibody titer. Treatment with a compound of formula (3) can be initiated before a surge in HHV-6 antibody titer to prevent an HHV-6 reactivation, or it can be initiated once a surge in antibody titer has been detected, to reduce the severity or duration of a reactivation event and to reduce the likelihood of complications associated with the reactivation, such as those discussed extensively herein.

In another aspect, the compound of formula (3) is used to treat a viral infection that is caused by a virus that is drug resistant. For example, the virus can be a strain that is at least about 5-fold less sensitive than wild type virus to an antiviral selected from ganciclovir, cidofovir, maribavir, acyclovir, penciclovir, and foscarnet.

In another aspect, the invention provides a method to prevent early-stage replication of a herpesvirus, by administering an effective amount of a compound of formula (3), such as artesunate to a subject. This method is useful to provide early intervention for a subject exposed to a herpesvirus, and may slow onset of or reduce the severity of an infection from the herpesvirus. The method is also useful to protect a subject from the effects of an abortive infection with a herpesvirus, such as HHV-6: even before an infection becomes well-established, it can trigger adverse reactions such as reactivation of another viral infection and significant detrimental immune reactions by producing the proteins associated with early, intermediate early, and late (E, IE and L) replicative stages. The method can also be used to reduce or prevent reactivation of a latent HHV-6 infection, or a secondary effect associated with reactivation of a latent HHV6 infection. Because the compounds of the invention inhibit replication at a very early stage of the replicative process, they reduce the production of E, IE and L stage viral proteins; the methods of the invention minimize the extent of such effects, and thereby reduce or prevent occurrence of disorders associated with IE, SITH, or other early-stage proteins associated with HHV-6 infection, or reactivation.

The methods are also useful for preventing a latent infection from being reactivated by triggering events such as exposure to a virus, bacterium or chemical agent that can trigger reactivation. Latent infections with herpesvirus can remain dormant for long periods of time; subjects having a latent herpesvirus infection may thus be in remission. Subjects in remission, however, remain at risk for reactivation. Because compounds of formula (3) act at such an early stage of viral replication, they are especially useful for maintaining remission in a subject having a latent herpesvirus infection.

Thus in another aspect, the methods provide a way to treat or manage a latent herpesvirus infection. This method may include intermittent treatments, such as once per month or once per week, with a compound of formula (3), to help maintain a subject in a state of remission. The method may also include administering an effective amount of a compound of formula (3) to a subject in remission when the subject has experienced an event that is likely to trigger reactivation, or when the subject is about to experience such an event. For example, a subject having a herpes virus infection that is in remission may be treated with a compound of formula (3) prior to or in preparation for surgery, chemotherapy or radiation therapy, if the treating physician considers these likely to trigger reactivation of the herpesvirus infection. A subject having a herpes virus infection that is in remission may be treated with a compound of formula (3) upon becoming pregnant. A subject having a herpes virus infection that is in remission may be treated with a compound of formula (3) following an exposure to a harmful chemical or biological agent. A subject having a herpes virus infection that is in remission may be treated with a compound of formula (3) when diagnosed with a separate viral or bacterial or fungal infection. A subject having a herpes virus infection that is in remission may be treated with a compound of formula (3) when diagnosed with a cancer or an autoimmune disorder. The cancer can be, for example, oral carcinoma, lymphoma (e.g., Hodgkins lymphoma), cervical or colorectal cancer. A subject having a herpes virus infection that is in remission may be treated with a compound of formula (3) after experiencing a physical trauma that is elicits a strong non-specific immune response, e.g., a broken bone or injured organ that requires surgical treatment, or following an organ or tissue transplant procedure. Because compounds such as artemisinin, dihydroartemisinin, artemether, artemotil, artelinic acid, and artesunate have excellent safety for use in humans, they can be administered prophylactically in these situations to prevent occurrence of serious complications that are likely to arise if a latent infection with a herpes virus is reactivated in a subject experiencing the physical stresses associated with events such as these.

In another aspect, a compound of formula (3) is administered to a subject in an effective amount, to treat or prevent a condition that is exacerbated by reactivation of HHV-6. This condition can be selected from, for example, epilepsy, MS, CFS, bipolar disorder, DIHS, FSE, status epilepticus, seizure, MTLE, encephalitis, pneumonitis and liver failure. In some embodiments, the subject for such treatment is a transplant patient, especially a recipient of a stem cell transplant. In subjects having tissue or organ transplants, the condition to be treated or prevented can be a complication selected from memory loss, fatigue, cognitive deficits, mephropathy (kidney transplant), GVHD, bone marrow suppression, CNS dysfunction, hepatitis, tissue rejection, susceptibility to fungal disease, amnesia, or immune suppression. In some embodiments, the subject is tested for the presence of HHV-6 virus or antibodies prior to such treatment to ensure that the subject is at risk for problems caused by reactivation of HHV-6.

The antiviral activity of the artemisinins appears to be associated with an effect on cellular activities involved in viral replication rather than a direct effect on the virus. Accordingly, these compounds seem to exhibit broad-spectrum antiviral activity, which affects a number of human and animal herpesviruses, such as HCMV, HHV-6, HSV-1 and Epstein-Barr virus (EBV), as well as RCMV and MCMV; see FIG. 2b. While some drug-resistant strains are slightly less sensitive to artemisinin derivatives, most virus variants showing resistance towards classical anti-herepsviral drugs (e.g. ganciclovir) are still quite sensitive to, e.g., artesunate. Particularly HHV-6, which is highly important clinically (as mainly recognized by recent studies, while frequently underestimated in earlier reports) and difficult to treat with classical anti-herpesviral drugs, shows a clear sensitivity to artesunate (FIGS. 1 and 2). Accordingly, the compounds of formula (3) are useful to treat a viral infection caused by a virus that is resistant to conventional antiviral drugs, such as ganciclovir, cidofovir, maribavir, acyclovir, penciclovir, and foscarnet; in some embodiments, the virus is HHV-6 or HHV-7. Thus the compounds of the invention can be administered to treat infections in subjects where at least one of these conventional therapies has stopped working, or where the viral strain can be identified as one wherein the strain is at least about 5-fold less sensitive in vitro to at least one of these antivirals than is the wild type virus.

Methods for ascertaining when artemisinin derivatives or compounds of formula (3) will be effective are well known in the art. For example, one can typically isolate a virus from a subject to be treated and determine whether the virus is sensitive to a particular compound of interest. Methods for assessing sensitivity to specific compounds such as artemisinin derivatives are known and are described herein. Where the virus replication or survival is inhibited by at least about 50% over 24 hours by a concentration of about 20 micromolar or less of the compound of interest, the virus is considered sensitive enough to the compound to be treated thereby, and that compound is suitably effective for use in treating the subject having the particular viral infection. In some embodiments, the virus is considered sensitive enough for treatment by a compound that affords 50% inhibition at a concentration of about 10 micromolar or less. Such experimentation is believed to be within the ordinary skill in the art. Accordingly, in some embodiments, the invention includes a method to treat a subject having a viral infection, wherein the method includes a step of determining whether the virus is sensitive to the compound of formula (3) prior to administering the compound of formula (3). The testing of the virus does not need to be done in some circumstances, of course; where the virus is known to be sensitive to a particular compound of formula (3), it is sufficient to characterize the virus by conventional methods to determine that it is a sensitive strain before administering a compound of formula (3). Alternatively, it may be sufficient to diagnose the subject as one having a titer of antibody to HHV-6 virus that is higher than expected for the subject's age, or the subject may be diagnosed as suitable based on demonstrated presence of DNA of an HHV virus such as HHV-6A or HHV-6B.

Artesunate acts at a very early stage of viral replication, thereby inhibiting the synthesis of non-structural viral proteins of the immediate early, early and late (IE, E and L) replicative stages and, as a consequence, the production of infectious progeny virus is inhibited. It also is believed to inhibit at an earlier stage of the life cycle of the virus relative to currently used antivirals, and thus reduces physiological effects of abortive infections that current antivirals cannot. Accordingly, in some embodiments the compound of formula (3) is administered at a very early stage post-infection, and may be administered prophylactically. Accordingly, in one embodiment, the invention includes use of a compound of formula (3) to prophylactically treat a subject who has been exposed to a virus, such as a herpes virus, even before symptoms appear. In one embodiment, the invention provides a method to treat a subject such as a medical practitioner, who has received an accidental exposure to material containing or suspected to contain live virus. Examples of such exposure include exposure to the blood of a subject having a viral infection, such as exposure from an accidental needle stick with a needle used to inject a subject having a viral infection.

The absence (or very rare occurrence) of severe side effects upon artesunate clinical treatment qualifies artesunate as a novel antiviral drug for use in prophylaxis, pre-emptive therapy as well as treatment of herpesviral infections. An important example for the use of artesunate, particularly in long-term regimens, is the antiviral treatment of patients after allogeneic stem cell transplantation suffering HHV-6-associated disease. Such patients have a compromised immune system (the subject is immunosuppressed) due to either direct effects of an underlying condition or the immunosuppressive drugs that are typically used to prevent tissue rejection. They are thus particularly susceptible to viral infections or fungal infections, and to reactivation of latent infections such as HHV-6, and conventional antiviral therapies (e.g., nucleoside mimics) are often contra-indicated in such patients due to adverse effects. The compounds of formula (3) are particularly useful for treating such patients, including subjects who have recently undergone stem cell transplantation, or who are currently undergoing stem cell transplantation, or who are about to undergo stem cell transplantation.

Drug resistance among herpesviruses is a widely recognized problem, and so far, there is no evidence that artemisinin or its derivatives produce resistance. Indeed, isolates of HHV-6 that are resistant to foscarnet and ganciclovir have been found and shown to contain mutations in genes associated with early viral replicative stage. Thus in one embodiment, a compound of formula (3) can be administered along with another antiviral to reduce occurrence of resistance to the other antiviral, and also to decrease the likelihood of resistance to the artemisinins. In order to reduce the potential occurrence of resistance development, and to increase effectiveness of the treatment methods, in another aspect the invention provides a method to treat a viral infection comprising administering a compound of formula (3) in combination with another conventional antiviral such as, for example, ganciclovir, cidofovir, maribavir, acyclovir, penciclovir, or foscarnet. This co-administration may be achieved by admixing a compound of formula (3) with another antiviral, in a composition that contains an effective amount of each of the two components; alternatively, the two medications can be administered concurrently but as separate compositions.

Alternatively, the method can be practiced by administering the compound of formula (3) to a subject within about 24 hours either before or after administration of a known antiviral compound. Typically, the compound of formula (3) and the antiviral compound will be administered within a time period of about 4 hours. While the compound of formula (3) and the antiviral compound can be administered by the same method, such as by admixing the two in a solution or suspension for a single injection, they can also be administered by different routes, provided an effective amount of each is administered by a route that is suitable for the specific compound being administered.

Similarly, the compounds of formula (3) can be used advantageously in patients who have herpesvirus infections that demonstrate resistance to treatment with other antivirals, such as, for example, ganciclovir, cidofovir, maribavir, acyclovir, penciclovir, or foscarnet.

### BRIEF DESCRIPTION OF THE DRAWINGS

FIG. 1 shows Inhibition of HHV-6A early protein production (p41/38) by ART. HSB-2/U1102 cells were cultivated in the presence of the drugs indicated for 3 days. Western blot analysis was performed by the use of MAb-05 (an antibody that recognizes P41/38) indicating the inhibitory effect of ART. Panels A and B show two representative results of this experiment. Samples were produced in duplicates as shown by two identical lanes of each test panel.

FIG. 2 shows Antiviral activity of artesunate (ART). Panel (a): ART sensitivity of HHV-6A, EBV and HCMV shown by immunofluorescence and plaque reduction assays. Panel (b): Summary of artesunate sensitivity of herpesviruses.

### DETAILED DESCRIPTION

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the ring or chain being described.

Typically, the alkyl, alkenyl and alkynyl substituents of the invention contain 1-10C (alkyl) or 2-10C (alkenyl or alkynyl). Alternatively, they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

While “alkyl” as used herein includes cycloalkyl and cycloalkylalkyl groups, the term “cycloalkyl” may be used herein to describe a carbocyclic non-aromatic group that is connected via a ring carbon atom (i.e., its open valence for connecting to a molecule is on a ring carbon), and “cycloalkylalkyl” may be used to describe a carbocyclic non-aromatic group that is connected to the molecule through an alkylene linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom (—C(O)—), and heteroacyl refers to the corresponding groups wherein at least one carbon other than the carbonyl carbon has been replaced by a heteroatom chosen from N, O and S. The other open valence of the carbonyl is available to connect the acyl group or heteroacyl group to a base molecule. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 alkenyl, C2-C8 alkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups, where the alkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups, or it includes an optionally substituted phenyl ring or C5-C6 monocyclic heteroaryl and a C1-C4 heteroalkylene that is unsubstituted or is substituted with one or two C1-C4 alkyl groups, where the alkyl groups can optionally cyclize to form a ring such as cyclopropane, dioxolane, or oxacyclopentane.

Where an arylalkyl group is described as optionally substituted, the substituents may be on either the alkyl portion or on the aryl or heteroaryl portion of the group. The substituents optionally present on the alkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Alkylene” as used herein refers to a divalent hydrocarbyl group; because it is divalent, it can link two other groups together. Sometimes it refers to —(CH₂)ₙ— where n is 1-8 and preferably n is 1-4, though where specified, an alkylene can also be substituted by other groups, and can be of other lengths. The open valences of an alkylene need not be at opposite ends of a chain. Thus —CH(Me)- and —C(Me)₂- are also included within the scope of the term ‘alkylenes’, as are cyclic groups such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. In some embodiments, the number of substituents permitted on a group is equal to the number of carbon atoms in the group. Where an optional substituent is attached via a double bond, such as a carbonyl oxygen (═O), the group occupies two available valences, so the total number of other substituents that may be included is reduced according to the number of other available valences.

“Halo”, as used herein includes fluoro, chloro, bromo and iodo. Fluoro and chloro are often preferred.

“Pharmaceutically acceptable salt” as used herein refers to a protonated or deprotonated form of a compound, such as a compound of formula (3), that is accompanied by a counterion, where the counterion is not harmful to a subject to be treated. Many counterions suitable for inclusion in pharmaceutically acceptable salts are known in the art. In many embodiments of the compounds of formula (3), the compound comprises a carboxylic acid; for such compounds, a salt can be formed by deprotonation of the carboxylic acid to form a carboxylate. The carboxylate will be accompanied by a counterion, and for making a pharmaceutically acceptable salt, the counterion is selected to have minimal toxicity or adverse effect on the subject to be treated. Examples of counterions for pharmaceutically acceptable salts of such carboxylates or other deprotonated species include, but are not limited to, so sodium, magnesium, potassium, calcium, iron, zinc, ammonium, alkylammonium, imidazolium, and the like.

“Subject” for the purposes of the present invention includes humans and other animals, particularly mammals. Thus the methods are applicable to both human therapy and veterinary applications. In certain embodiments the subject is a mammal, and in a preferred embodiment the subject is human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age and weight of the patient to be treated, and the like. The therapeutically effective amount can be determined routinely by one of ordinary skill in the art having regard to their knowledge and to this disclosure.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal gland: neuroblastoma. Thus, the term “cancerous cell” as provided herein, includes a cell afflicted by any one of the above-identified conditions.

“Treating” or “treatment” as used herein covers the treatment of a disease-state in a human, which disease-state is characterized by abnormal cellular proliferation, and invasion and includes at least one of: (i) preventing the disease-state from occurring in a human, in particular, when such human is predisposed to the disease-state but has not yet been diagnosed as having it; (ii) inhibiting the disease-state, i.e., arresting its development; and (iii) relieving the disease-state, i.e., causing regression of the disease-state. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

In one aspect, the invention provides methods to treat human herpes virus infections, or to prevent or reduce the severity or probability of a reactivation of a latent herpes virus infection. In some aspects, it is used to prevent or reduce the likelihood of an adverse condition associated with reactivation of latent HHV-6 infection. Such adverse conditions include, but are not limited to, myocarditis, certain cancers, encephalitis, various neurologic disorders (e.g., epilepsy, MS, CFS), DIHS, and certain psychiatric disorders. The methods comprise administering an effective amount of a compound of formula (3) or a pharmaceutical composition containing such a compound of formula (3), to a subject in need of such treatment. The compounds of formula (3) include:

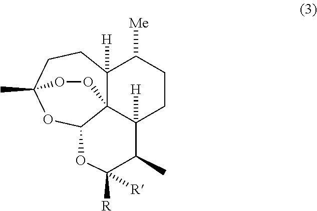

wherein R and R′ taken together form a carbonyl (═O),

or

wherein one of R and R′ is H,

and the other one of R and R′ is —OA,

wherein A is H or an optionally substituted group selected from alkyl, aryl, heteroaryl, arylalkyl, and heteroarylalkyl;

or a pharmaceutically acceptable salt or ester thereof.

In some embodiments of the compound of formula (3), R′ is H.

In some embodiments of the compound of formula (3), A is H or C1-C6 alkyl, or a substituted C1-C6 alkyl. Where A is a substituted alkyl, it is sometimes substituted with a carbonyl (═O), and in some embodiments —O-A is a group of the formula —O—C(═O)—Z, where Z is a C1-C4 alkyl that can be substituted, such as with one or more halo, —OH, or C1-C4-alkoxy groups. These acyl groups of the formula —O—C(═O)—Z are prodrugs that release a compound of formula (3) where A is H when exposed to the ubiquitous esterase activities in a living organism.

In some embodiments, A comprises a carboxylic acid. In specific examples, A can be —(CH₂)ₙ—COOH; —C(═O)—(CH₂)ₙ—COOH; or —(CH₂)ₙ—Ar, wherein n is 1-4 and Ar represents an optionally substituted aryl or heteroaryl ring. In some embodiments, Ar is substituted with a carboxylic acid (COOH) group.

In some embodiments, the foregoing methods are used to treat an infection caused by a beta herpes virus, or one associated with reactivation of a beta-herpes virus. In certain embodiments, the virus involved is HHV-6 or HHV-7; in specific embodiments of special interest, it is HHV-6A or HHV-6B. In some embodiments, the virus is a drug-resistant type, such as one resistant to an antiviral drug selected from ganciclovir, cidofovir, maribavir, acyclovir, penciclovir, and foscarnet. Resistance, in this context, means the virus is at least 5-fold less sensitive to one or more of these drugs than a wild-type isolate of the same virus.

The compound of formula (3) can take various forms, but in some embodiments, this compound is selected from the group consisting of artemisinin, dihydroartemisinin, artemether, artemotil, artelinic acid, and artesunate.

In some embodiments of the invention, a subject is selected for treatment based on an elevated antibody titer for an antibody associated with the particular herpes virus. An elevated antibody titer (e.g., a titer above about 1:320 or above 1:640, or in the top 25% to 15% of the normal level for such subjects; or a titer that is at least one or at least two dilutions above the mean titer for the subject's cohort) identifies the subject as one experiencing or at risk for an infection by the particular virus, or experiencing or at risk for reactivation of that virus. A subject having other risk factors in combination with an elevated HHV-6 antibody titer may be especially suitable for treatments described herein. For example, a subject who has genetic risk factors for epilepsy or MS, or who is receiving or is about to receive an immunosuppressant treatment, or who is receiving a drug associated with DIHS, may be identified as suitable for the treatments described herein.

In some embodiments, the virus may be tested to determine if it is sufficiently sensitive to a compound of formula (3), before treatment is initiated, and a compound of formula (3) may be administered upon showing that the virus is sensitive to the compound of formula (3).

The methods described herein are appropriate for treatment of infections or conditions associated with herpes viruses, particularly beta herpes virus or gamma herpes viruses. For example, the methods may be used to treat a viral infection or associated disorder where the virus is Epstein-Barr virus, HHV-8, HHV-3, Kaposi sarcoma-associated herpes virus, or varicella zoster virus.

In certain aspects, the invention provides a method to treat or prevent a condition that is caused or exacerbated by reactivation of a herpesvirus, and the method comprises administering to a subject in need of such treatment an effective amount of a compound of formula (3). A variety of conditions that can be caused or exacerbated by reactivation of a latent herpes virus infection are discussed above: specific examples include certain cancers (oral carcinoma, cervical cancer, colorectal cancers, and lymphomas), DIHS, MS, CFS, epilepsy, other neurological disorders associated with HHV-6, encephalitis, myocarditis, GVHD, tissue or organ rejection, and the like. In some embodiments, the condition is selected from a neurological disorder, a cancer, myocarditis, a transplant related complication, encephalitis, and a psychiatric disorder.

Suitable subjects for these treatments can be identified by a person of ordinary skill based on the disclosures herein. In some embodiments, the subject is a transplant patient, particularly a stem cell recipient. In other embodiments, the subject is one receiving or about to receive an immunosuppressant drug, or a drug that is associated with DIHS (e.g., an anticonvulsant).

In some embodiments, the compound of formula (3) is administered as a monotherapy to a subject needing the treatments described above. In other embodiments, a compound of formula (3) is administered concurrently with another antiviral drug, e.g., in combination with an effective amount of another antiviral selected from ganciclovir, cidofovir, maribavir, acyclovir, penciclovir, and foscarnet. In some embodiments, because of the particular mode of action of compounds of formula (3), the methods described herein are used to treat a subject infected with a virus that is resistant to one or more common antivirals. In such embodiments, the two drugs may be administered separately or they may be combined into a single composition.

In another aspect, the invention provides a method to inhibit production of viral replication-associated proteins in a subject, wherein the proteins are associated with a herpesvirus infection. Inhibition of the production of these proteins can slow or halt progression of an underlying HHV-6 infection, for example. These methods comprise administering to a subject in need of such treatment an effective amount of a compound of formula (3), or one of the specific compounds identified above, or a combination of such compound with another antiviral drug such as ganciclovir, cidofovir, maribavir, acyclovir, penciclovir, or foscarnet. Compounds of formula (3) particularly suited for use in this and other methods disclosed herein include artemisinin, dihydroartemisinin, artemether, artemotil, artelinic acid, and artesunate.

In certain of these embodiments, the subject selected for treatment is one who exhibits a genetic risk for at least one condition that is associated with reactivation of latent HHV-6 infection. For example, a subject having a genetic risk factor for MS, CFS, DIHS, epilepsy, or a cancer or neurological or psychiatric disorder that is associated with HHV-6 reactivation (such as those disclosed herein) may be selected for treatment with the methods disclosed herein. The methods thus provide away to inhibit an adverse effect associated with reactivation of a latent HHV-6 infection.

These risk factors may be used alone to select a suitable subject, or they may be used in combination with risk factors specific for a particular viral infection. For example, a subject's HHV-6 antibody titer may be used to identify the subject as one at risk for an HHV-6 infection, or one having an HHV-6 infection that is latent. An elevated titer of antibodies to HHV-6 may be used to diagnose a subject as at risk for reactivation of a latent HHV-6 infection, where the titer is above a set level (e.g., 1:320 or 1:640 for an adult), or above statistical norms for the subject's age and gender.

Preferably, an antibody titer for such a diagnosis or evaluation will be measured using IFA (indirect fluorescent antibody) methods, which are more quantitative and reliable than ELISA, and preferably the titer selected as a cut-off value will be based on normal values measured by the same technique. For example, a cut-off for this diagnosis may be at least one dilution (2×) above the median for healthy subjects similar to the patient. In some embodiments, the cut-off will be two dilutions (4×) above a median value for healthy subjects. For example, a subject may be identified as suitable for treatment with the methods disclosed herein when his or her antibody titer is in the top 25% range for the subject's cohort; or within the top 15% range for the subject's cohort. In certain instances, the subject is diagnosed as suitable for treatment based on antibody titer if the subject's antibody titer is at least one dilution (i.e., 2×) higher than that for age and gender-matched healthy subjects.

Alternatively, the subject may be tested for the presence of one or more proteins or nucleic acids associated with a particular virus; for example, a subject in which IE1, IE2, or SITH is detected can be identified as one in need of treatment for an HHV-6 infection, or for a condition associated with, caused by or exacerbated by an HHV-6 reactivation. It has been shown, for example, that spinal fluid and plasma of subjects diagnosed with chronic fatigue syndrome often show high levels of HHV-6 markers via PCR (34% in spinal fluid, 48% in plasma); thus this provides a way to identify a subject for treatment of HHV-6 associated disorders. Alternatively, the subject may be one who has or is about to receive medications known to be associated with adverse effects in a subject infected with HHV-6. In some embodiments, the progress of treatment of a subject with the methods described above can be monitored by monitoring the level of antibody titers for proteins associated with HHV-6, for example.

In addition to the methods described herein, the invention provides compounds of formula (2) or formula (3) for use in the treatment of certain herpes virus infections, such as HHV-6, HSV-1, EBV, VZV, and the like, and for use to prevent or inhibit viral reactivation in persons infected with one of these viruses, and to prevent or reduce the likelihood of occurrence of various disorders associated with the viral activity or reactivation of one of these viruses.

Specific details useful for the practice of the inventions described herein are set forth below.

Methods of Administration

Administration of the compounds of the invention, or their pharmaceutically acceptable salts, in pure form or in an appropriate pharmaceutical composition, can be carried out via any of the accepted modes of administration of agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, gel, ointment, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages. In some embodiments, the dosage form is a solution that is suitable for administration by injection, such as intramuscular, subcutaneous, or intravenous injection. Such dosage forms may be administered in a single bolus or by an infusion, or by other methods known in the art.

Artemisinin and its derivatives are often suitably administered orally, as a tablet or powder, or as a solution or suspension. In some embodiments, the compound of formula (3) is preferably delivered by injection. In some embodiments, topical administration is appropriate, such as where the compound of formula (3) is used to alleviate a localized lesion on the skin. In some embodiments, it can be administered as a suppository.

In some embodiments, the compounds or compositions of the invention are administered about once per week. In some embodiments, they are administered about once per day, or at least once per day. In some embodiments, the compounds and compositions may suitably be administered in two or more dosages per day. Selection of the timing and frequency of administration is generally within the level of an ordinarily skilled practitioner.

While an HHV-6 titer of 1:1280, 1:640 or 1:320 in a child or adolescent might be perfectly normal, a titer of this level is rare in an adult and could be a sign of chronic infection in an adult with clinical symptoms. Thus in certain embodiments, the treatments disclosed herein are initiated in response to an elevated HHV-6 titer that is abnormally high for the subject relative to the subject's peers (considering demographic factors such as age, gender, etc.), and in some embodiments a treatment described herein is monitored by checking the subject's HHV-6 titer. For example, where symptoms consistent with a disorder associated with HHV-6 are observed, the subject may be tested for a relatively high titer of HHV-6 antibodies (e.g., higher than 1:1280, 1:640 or 1:320 in an adult) for his age; and if the HHV-6 antibody titer is relatively high, treatment with the methods disclosed herein may be initiated. Treatment may then be continued for a period sufficient to produce a response, and monitored by tracking any change in the subject's HHV-6 antibody titer.

The compositions will include a conventional pharmaceutical carrier or excipient and a compound of the invention as the/an active agent, and, in addition, may include other medicinal agents, pharmaceutical agents, carriers, adjuvants, etc. Compositions of the invention may be used in combination with anticancer or other agents that are generally administered to a patient being treated for cancer. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Compositions suitable for parenteral injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One preferable route of administration is oral, using a convenient dosage regimen that can be adjusted according to the degree of severity of the disease-state to be treated. Formulations and dosages for treatment of malaria provide a sound starting point for selection of a suitable dosage for treating herpesvirus infections, as these have been shown to be safe in humans and to be effective. Oral administration of such compounds has been proven effective in treatments of malaria, demonstrating that it is an effective means for administering compounds of formula (3) to achieve effective concentrations in vivo.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 1 to about 100 mg per kilogram of body weight per day, or from about 1 to about 20 mg/kg per day, is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

Artemisinin and many of its derivatives are available from commercial sources, and methods for making its derivatives such as those described herein are known in the art.

The following examples are offered to illustrate but not to limit the invention.

### EXAMPLE 1

### Generation of a HHV-6A-Positive Cell Line and Detection of Viral Proteins

HSB-2 were used for the infection with HHV-6A and prototype strains U1102. An aliquot of the virus-infected cells (stored at −80° C.) was thawed, washed and freshly added to an excess of uninfected cells. After addition of medium, cells were cultivated at 37° C. in a CO₂ incubator. After appearance of a virus-induced c.p.e. (cytopathic effect) (app. 5 days), HSB-2/U 1102 cells were passaged and used as permanently infected cell line and used for the analysis of antiviral drugs. Uninfected HSB-2 cells serve as a control to detect any cell alterations due to other infections, and no c.p.e. were observed in uninfected cells.

The production of viral proteins can be detected by the HHV-6-specific antibodies available from the HHV-6 Foundation's repository. MAb-05 was positive in Western blot detection procedures, while all other analyzed antibodies were negative (i.e. they lacked HHV-6-specific signals in Western blot and indirect immunofluorescence tests). MAb-C5 detected the viral early protein p41/38 in HSB-2/U1102 cells at various time points of cultivation. Positive signals were obtained between 2 and 4 weeks post infection. Signal quantities increased up to the time point of 4 weeks, but decreased later on and seemed to underlie fluctuations which might reflect varying viral productivity in these permanently infected cells (data not shown).

Note that MAb-C5 recognizes P41/38, and is specific for HHV-6A-infected cells. This protein first appears 12-18 hours after viral infection, thus it is a very early indicator of the presence of infection.

### EXAMPLE 2

### Inhibition of p41/38 Protein Production in HSB-2/U1102 Cells by Artesunate at an Early Stage of Replication

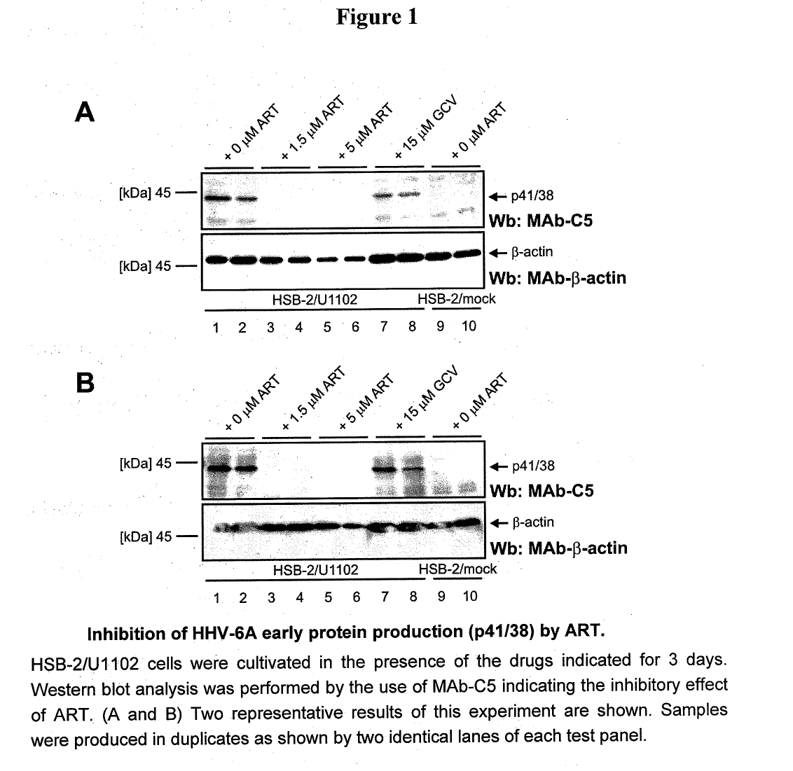

HSB-2/U1102 cells were treated with various concentrations of artesunate (ART) and cultivated for 3 days. Thereafter, cells were lysed and analyzed for the presence of viral p41/38 by SDS-PAGE and Western blot detection. As a clear-cut result, levels of p41/38 were diminished by ART treatment below the detection limit, while a cellular control protein (β-actin) was not affected or showed only marginal alterations (FIGS. 1A and B, representing two equivalent settings of this experiment). 1.5 μM and 5 μM of ART showed identical results indicating that the inhibitory potential was optimal in a micromolar range. Interestingly, the reference drug ganciclovir (GCV) did not show a similar degree of inhibition. This might indicate different modes of action, and it is believed that GCV does not inhibit production of early protein p41. While GCV is known as an inhibitor of herpesviral DNA synthesis (inhibiting the late phase of replication, but allowing viral IE/E protein production), ART appears to act at a much earlier stage. This is consistent with the findings described for the inhibition of human cytomegalovirus (HCMV) replication by ART (Efferth et al., 2002). In the case of HCMV, protein production is inhibited at all stages of the viral replication cycle, i.e. IE, E and L (immediate early, early and late), thus indicating a very early block. The findings that cellular transcription factors, such as NF-κB and SP1, are down-regulated by ART, are compatible with this idea and lead to the proposal of a mechanism of inhibition based on cellular activation pathways required for viral replication (Efferth et al., 2002; Wagner et al., 2006).

### EXAMPLE 3

### Inhibition of HHV-6 Early Protein Production

MOLT-3 cells are infected with HHV-6B, Z-29 variant, and cultured in the presence or absence of ART for 3 days. Western blot analysis is performed using mAb 6A5D12, which detects both HHV-6A and HHV-6B variants of p41, an Early Protein of HHV-6. ART at 1.5 μM or 15 μM blocks production of detectable amounts of p41, while p41 is detected in untreated controls. Accordingly, it is shown that ART interferes with progression of HHV-6 infection from the latent stage characterized by IE to an active stage where p41 would be produced.

### EXAMPLE 4

### Determination of IC50s

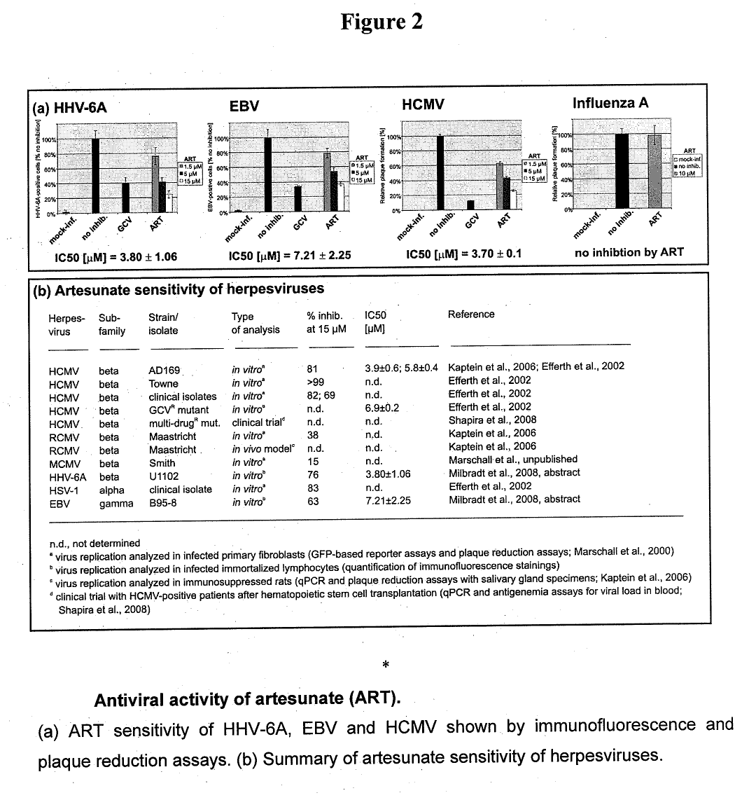

HSB-2/U1102 cells were treated with ART (concentrations as indicated), or GCV as a control, for 3 days before the cells were subject to chemical fixation and used for indirect immunofluorescence detection (using a pool of human HHV-6-positive antisera; FIG. 2a; left panel). HSB-2 were used as a mock-infected control. In parallel Epstein-Barr virus (EBV, strain B95-8)-infected Raji cells were also analyzed for ART sensitivity under the same experimental conditions. For quantifications, each sample (performed in duplicates) was used for microscopic counting of two comparable fields (i.e. resulting in four values). HHV-6-positive cells were determined in each field and the percentage towards infected cells without inhibitor was used to calculate the mean inhibitory concentration (IC50). Both viruses showed clear ART sensitivity, whereby the IC50 for HHV-6A (3.80±1.06 μM) was lower than for EBV (7.21±2.25 μM). As further controls, the sensitivity of human cytomegalovirus (HCMV, strain AD169) and human influenza A virus (Influenza A, strain WSN/33) was analyzed in parallel by the use of standard plaque reduction assays. While HCMV shows a marked ART-sensitivity, influenza A virus was not inhibited.

### EXAMPLE 5

### Inhibition of HHV-6 Early Protein Production

MOLT-3 cells infected with HHV-6B strain Z-29 are treated with ART, or with gancyclovir (GCV) as a control, for 3 days. Viral DNA synthesis is then tested by PCR. Cells are then fixed with acetone treatment. PCR shows that ART blocks viral DNA synthesis more effectively than does GCV. In addition, an immunofluorescence antibody test for HHV-6 proteins showed ART inhibited formation of HHV-6 viral proteins.

### EXAMPLE 6

### Inhibition of HHV-6 IE Protein Production

Both HHV-6A and HHV-6B generate IE protein before viral DNA synthesis is detected; IE appears within 4-6 hours after infection, and appears to be crucial to viral replication. HSB-ML, a cell line carrying the latent genome for HHV-6A, is infected with GS strain of HHV-6A. The cells are then exposed to ART and progression of the infection will be monitored by detection of U94 or of latent proteins detectable by antibodies, e.g., by using an antibody specific for p37 (one of the IE proteins), and one specific for SITH, another latent stage HHV-6 protein. ART is found to inhibit formation of U94, p37, and SITH, demonstrating that it inhibits infection at a very early stage in the infection process, and delays or prevents formation of at least some of the IE proteins. SITH is found in 76% of bipolar patients, 71% of CFS patients with neurological conditions, and 63% of depressed patients were found to have antibodies to SITH, while none of the control subjects had detectable amounts of this antibody. Kobayashi, et al. SITH production is thus associated with these neurological conditions, and ART is able to reduce the risk of their occurrence by inhibiting HHV-6 infection at an early stage, before SITH production.

### EXAMPLE 7

### Lack of Cytotoxic Side-Effects

Infected and uninfected HSB-2 cells were analyzed in a standard procedure for the detection of putative drug-induced apoptosis signals (Caspase-Glo™ 3/7 Assay; Promega). ART was analyzed at concentrations between 0.5 μM and 15 μM as indicated. As an apoptosis-inducing positive control, etoposide was used in parallel at the concentrations suggested by the manufacturer (Sigma). Clearly, apoptosis signals induced by ART (3 days incubation) were very low and did not markedly increase at concentrations below 15 μM. Second, a cytotoxicity assay was performed (CytoTox 96 Non-Radioactive Cytotoxicity Assay; Promega) which determines the lactate dehydrogenase (LDH) release from necrotic cells. HHV-6-negative T-lymphocytes (CEMx174) and B-lymphocytes (Raji) were used as reference cells lines and analyzed for cytotoxicity signals produced by the incubation of ART (3 days). Generally, the induction of cytotoxicity by ART was very low in both cell types up to the concentration of 90 μM. Only some increase was noted for the high concentrations. Based on these results, a superimposition of the determined antiviral effects effects by unwarranted cytotoxic side-effects can be mostly excluded.

### EXAMPLE 8

### Broad Anti-Herpesviral Activity of Artesunate

Previously, the strong anti-cytomegaloviral activity of ART was published by our group (Efferth et al., 2006; Kaptein et at, 2006). In these studies, the inhibitory potential of ART towards various cytomegaloviruses (human and animal CMVs) was described, both in cell culture models and in infected animals (Kaptein et al., 2006). Importantly, the antiviral activity is not restricted to distinct viral strains but is also seen for clinical isolates of HCMV and mutants with resistance against conventional antiviral drugs, such as ganciclovir and cidofovir (New data show that also other herpesviruses from all subfamilies (alpha, beta and gamma) are sensitive towards ART, namely EBV, HSV-1 and HHV-6. This finding leads to the suggestion that ART displays a broad anti-herpesviral activity, whereby the ART sensitivity of individual herpesviruses varies within the low micromolar range. Since the mode of action is not described in detail so far, the basis for sensitivity remains speculative. However, the current status of investigations strongly encourages the use of ART in anti-herpesviral therapy. Of note, artesunate has been in use for the treatment of plasmodium infections (severe malaria) for a long time and recently a novel artesunate-amodiaquine drug combination was approved for uncomplicated malaria, demonstrating substantial safety margin for use of artesunate in therapy. Concerning the chances of ART in anti-herpesviral therapy, particular interest would be in clinically complex situations which cannot be resolved by conventional antiviral treatment, such as organ and bone marrow transplantations with life-threatening herpesvirus reactivations, where conventional antivirals such as ganciclovir cannot safely be used because they would be toxic to the newly introduced tissue or stem cells.

### EXAMPLE 9

### Inhibition of Drug-Resistant HHV-6 Isolates

A number of drug-resistant HHV-6 isolates are known, and show resistance to antivirals including gancyclovir, cidofovir, foscarnet, and other antiviral drugs. The following Table describes a few known drug-resistant HHV-6B isolates. ART will be tested on HHV-6B isolates listed in the table which are resistant to PFA (foscarnet), GCV (gancyclovir) and CDV (cidofovir), in MT4 cells. These isolates are from patients who were treated for a long period of time with the specified antivirals. The references are included below. These HHV-6B isolates are available from the HHV-6 Foundation Repository.

TABLE
Characterization of selected viruses stocks derived from wild-type HST strain
(HHV-6 B) on MT4 cells (Bonnafous et al., 2008; Bonnafous et al., 2007)

| Viral strain | | | | | | Date of stock^{c} | Highest concentration |
|---|---|---|---|---|---|---|---|
| (parental | IC₅₀ ± SD (μM)^{a} [RI^{b}] of drug | | | pU38 | pU69 | (titre in | of antiviral used for |
| virus) | PFA | GCV | CDV | changes | change | TCID₅₀/ml) | selection |
| HST | 15.0 ± 7.0 | 3.1 ± 0.8 | 1.8 ± 0.9 | WT | WT | 10 Apr. 2009 | none |
| | | | | | | (10³) | |
| GCVR1 | 18.9 ± 5.4 | 25.9 ± 2.8 | 57.8 ± 27.6 | A961V | M318V | 10 Apr. 2009 | 128 μM of GCV |
| (HST) | [1.3] | [8.4] | [32.6] | | | (6 × 10²) | |
| GPFAR1 | 217.9 ± 17.3 | 25.7 ± 3.9 | 12.5 ± 4.4 | T435R, | M318V | 28 Nov. 2006 | 640 μM of PFA |
| (GCVR1) | [14.6] | [8.4] | [7.0] | C525S | | (10⁴) | |
| | | | | A961V | | | |
| CDVR1 | 17.8 ± 6.9 | 12.9 ± 4.3 | 213.2 ± 77.3 | R798I | WT | 28 Nov. 2006 | 256 μM of CDV |
| (HST) | [1.2] | [4.2] | [118.4] | | | (2 × 10³) | |

SD, standard deviation; PFA, foscarnet; GCV, ganciclovir; CDV, cidofovir; WT, wild-type; TCID₅₀, tissue culture infectious dose 50%
^{a}The IC₅₀ (inhibitory concentration 50%) was derived from the inhibition curve and defined as the concentration of antiviral drug that reduced the marker of virus replication (DNA copy number per cell) by 50% compared to that observed in the absence of the drug. Assays were performed in duplicate and data represent the mean of two to six independent experiments.
^{b}The RI (resistance index) was computed as the ratio of IC₅₀ of a given virus to HST. Significant values for RI (≧3) are noted in bold.
^{c}The date of stock is given in day/month/year.
Bonnafous, P., Boutolleau, D., Naesens, L., Deback, C., Gautheret-Dejean, A. and Agut, H., 2008. Characterization of a cidofovir-resistant HHV-6 mutant obtained by in vitro selection. Antiviral Res 77, 237-40.
Bonnafous, P., Naesens, L., Petrella, S., Gautheret-Dejean, A., Boutolleau, D., Sougakoff, W. and Agut, H., 2007. Different mutations in the HHV-6 DNA polymerase gene accounting for resistance to foscarnet. Antivir Ther 12, 877-88.

ART produces greater inhibition of viral infection in this test than the drug to which each isolate has developed resistance. This demonstrates that ART and compounds of formula (3) are useful to treat drug-resistant isolates of HHV-6, and suggests that combinations of ART or a compound of formula (3) can be used in combination with such known antivirals as PFA, GCV, and CDV to provide antiviral activity while reducing occurrence of resistance.

1.-35. (canceled)

36. A method to treat a herpes viral infection or a condition that is induced or exacerbated by a herpes viral activity, infection or protein, comprising administering to a subject in need thereof an effective amount of a compound of formula (3): 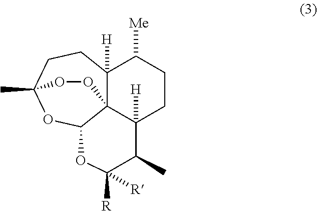
wherein R and R′ taken together form a carbonyl (═O),
or
wherein one of R and R′ is H,
and the other one of R and R′ is —OA,
wherein A is H or an optionally substituted group selected from alkyl, aryl, heteroaryl, arylalkyl, and heteroarylalkyl;
or a pharmaceutically acceptable salt or ester thereof.

37. The method of claim 36, wherein the viral infection is caused by a beta-herpes virus.

38. The method of claim 37, wherein the herpes virus is HHV-6 or HHV-7.

39. The method of claim 38, wherein the herpes virus is HHV-6A or HHV-6B.

40. The method of any one of the preceding claims, wherein the compound of formula (3) is selected from the group consisting of artemisinin, dihydroartemisinin, artemether, artemotil, artelinic acid, and artesunate.

41. The method of claim 37, wherein the viral infection is caused by a virus that is drug resistant.

42. The method of claim 41, wherein the virus that is drug resistant is at least 5-fold less sensitive than wild type virus to an antiviral selected from ganciclovir, cidofovir, maribavir, acyclovir, penciclovir, and foscarnet.

43. The method of claim 36, further comprising testing the virus causing the viral infection to determine if it is sufficiently sensitive to the compound of formula (3), and administering the compound of formula (3) to the subject only if the virus is sensitive to the compound of formula (3).

44. The method of claim 36, wherein R′ in the compound of formula (3) is H.

45. The method of claim 37, wherein the viral infection is caused by a gamma-herpes virus.

46. The method of claim 36, wherein the viral infection is caused by a virus selected from Epstein-Barr virus, HHV-8, and varicella zoster virus.

47. A method to treat a herpesvirus viral infection in a subject, said method comprising administering to a subject in need of treatment for a herpes viral infection an effective amount of a compound of formula (3): 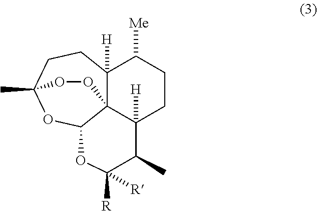
wherein R and R′ taken together form a carbonyl (═O),
or
wherein one of R and R′ is H,
and the other one of R and R′ is —OA,
wherein A is H or an optionally substituted group selected from alkyl, aryl, heteroaryl, arylalkyl, and heteroarylalkyl;
or a pharmaceutically acceptable salt or ester thereof;
in combination with an effective amount of another antiviral selected from the group consisting of ganciclovir, cidofovir, maribavir, acyclovir, penciclovir, and foscarnet.

48. The method of claim 47, wherein the viral infection is caused by a beta-herpes virus.

49. The method of claim 48, wherein the beta-herpes virus is HHV-6 or HHV-7.

50. The method of claim 49, wherein the virus is HHV-6A.

51. The method of claim 49, wherein the virus is HHV-6B

52. The method of claim 47, wherein the subject is a transplant patient.

53. The method of claim 52, wherein the subject is a stem cell transplant patient.

54. A method to inhibit an adverse effect associated with reactivation of a latent HHV-6 infection, comprising administering to a subject in need of such treatment an effective amount of a compound of formula (3).

55. The method of claim 54, wherein the compound is selected from the group consisting of artemisinin, dihydroartemisinin, artemether, artemotil, artelinic acid, and artesunate.

56. The method of claim 54, wherein the subject is one who has been diagnosed as being at risk of an adverse effect associated with reactivation of a latent HHV-6 infection.

57. The method of claim 56, wherein the subject is an adult having an HHV-6 antibody titer of at least 1:320.

58. The method of claim 57, further comprising monitoring the level of an HHV-6 antibody in the subject, or monitoring the presence or level of one or more HHV-6 proteins in the subject.
